(19) United States
(12) Patent Application Publication
(10) Pub. No.: US 2024/0081900 A1
(43) Pub. Date: Mar. 14, 2024

(54) CATHETER

(71) Applicant: Japan Lifeline Co., Ltd., Tokyo (JP)

(72) Inventors: Kohei SAKAKI, Tokyo (JP); Makoto KATO, Tokyo (JP); Ryo ISHIDA, Tokyo (JP)

(21) Appl. No.: 18/356,751

(22) Filed: Jul. 21, 2023

(30) Foreign Application Priority Data
Sep. 13, 2022 (JP) 2022-145456

(51) Int. Cl. A61B 18/14

(52) U.S. Cl. CPC A61B 18/1492; A61B 2018/00577

(57) ABSTRACT

A catheter includes an elongated body, a distal end side of which is inserted into a body, and an electrode provided around an axis of the elongated body and expandable in a direction intersecting the axis. The electrode in an expanded state includes an intermediate portion at least partially including a portion in which element wires spread like a mesh, a proximal end portion located closer to a proximal end side of the elongated body than the intermediate portion and in which the element wires gather, and a distal end portion located closer to the distal end side than the intermediate portion and in which the element wires gather. The intermediate portion includes a maximum portion where an outer dimension of the electrode is maximized. The maximum portion is located closer to the proximal end side than a center of the electrode in an axial direction of the elongated body.

### CROSS REFERENCE TO RELATED APPLICATIONS

This application claims the benefit of priority to Japanese Patent Application Number 2022-145456 filed on Sep. 13, 2022. The entire contents of the above-identified application are hereby incorporated by reference.

### TECHNICAL FIELD

The present disclosure relates to a catheter.

### BACKGROUND

A catheter is a member to be inserted into a body for diagnosis and treatment. For example, JP 2022-60361 A discloses a catheter having a spherical ablation electrode provided on its shaft.

### SUMMARY

Catheters for ablation always require improved performance such as usability and durability.

The present disclosure has been made in view of such circumstances, and an object thereof is to provide a technique for improving the performance of catheters.

One aspect of the present disclosure is a catheter. This catheter includes an elongated body at least a distal end side of which is to be inserted into a body and an electrode provided around an axis of the elongated body and expandable in a direction intersecting the axis. The electrode in an expanded state includes an intermediate portion at least partially including a portion in which element wires spread like a mesh, a proximal end portion which is located closer to a proximal end side of the elongated body than the intermediate portion and in which the element wires gather, and a distal end portion which is located closer to the distal end side than the intermediate portion and in which the element wires gather. The intermediate portion includes a maximum portion where an outer dimension of the electrode is maximized. The maximum portion is located closer to the proximal end side than a center of the electrode in an axial direction of the elongated body.

Another aspect of the present disclosure is also a catheter. This catheter includes an elongated body at least a distal end side of which is to be inserted into a body and an electrode provided around an axis of the elongated body and expandable in a direction intersecting the axis. The electrode in an expanded state includes an intermediate portion at least partially including a portion in which element wires spread like a mesh, a proximal end portion which is located closer to a proximal end side of the elongated body than the intermediate portion and in which the element wires gather, and a distal end portion which is located closer to the distal end side than the intermediate portion and in which the element wires gather. The intermediate portion includes a maximum portion where an outer dimension of the electrode is maximized. The electrode includes a proximal end-side region from the proximal end portion to the maximum portion and a distal end-side region from the maximum portion to the distal end portion. In a first region continuing from the proximal end portion in an axial direction of the elongated body within the proximal end-side region and a second region continuing from the maximum portion in the axial direction within the distal end-side region, the number of the element wires in the first region is less than the number of the element wires in the second region, and a thickness of the element wires in the first region is larger than a thickness of the element wires in the second region.

Yet another aspect of the present disclosure is also a catheter. This catheter is a catheter for pulsed electric field ablation and includes an elongated body at least a distal end side of which is to be inserted into a body and an electrode that is provided on the elongated body and generates a pulsed electric field. The electrode includes a core wire and a coating covering the core wire. The core wire contains a first metal whose standard electrode potential is lower than a standard electrode potential of hydrogen. The coating contains a second metal whose standard electrode potential is higher than the standard electrode potential of hydrogen and which is less likely to occlude hydrogen than the first metal.

Any combination of the above components and conversions of expressions of the present disclosure between a method, a device, a system, and the like are also effective as aspects of the present disclosure.

The present disclosure can improve the performance of catheters.

### BRIEF DESCRIPTION OF DRAWINGS

FIGS. 1A and 1B are side views of a catheter according to an embodiment.

FIG. 2 is a perspective view of an electrode.

FIG. 3 is a side view of an electrode.

FIG. 4 is a view illustrating a usage state of a catheter.

FIG. 5 is a cross-sectional view of an element wire that constitutes an electrode.

FIGS. 6A, 6B, 6C, 6D, 6E, and 6F are diagrams each illustrating a manufacturing process of an electrode.

### DESCRIPTION OF EMBODIMENTS

The present disclosure will be described below based on preferred embodiments with reference to the drawings. The embodiments do not limit the present disclosure and are illustrative. Not all features or combinations thereof described in the embodiments are necessarily essential to the present disclosure. The same or similar components, members, and processing operations illustrated in the drawings are denoted by the same reference numerals, and redundant descriptions are omitted as appropriate. The scales and shapes of the parts illustrated in each drawing are set for convenience to facilitate the explanation and should not be construed in a limited manner unless otherwise specified. When the terms “first,” “second,” and the like are used in the present description or claims, these terms do not mean any order or importance unless otherwise specified and are used for distinguishing a configuration from other configurations. Furthermore, some members that are not important in describing the embodiments in the drawings are omitted in illustration.

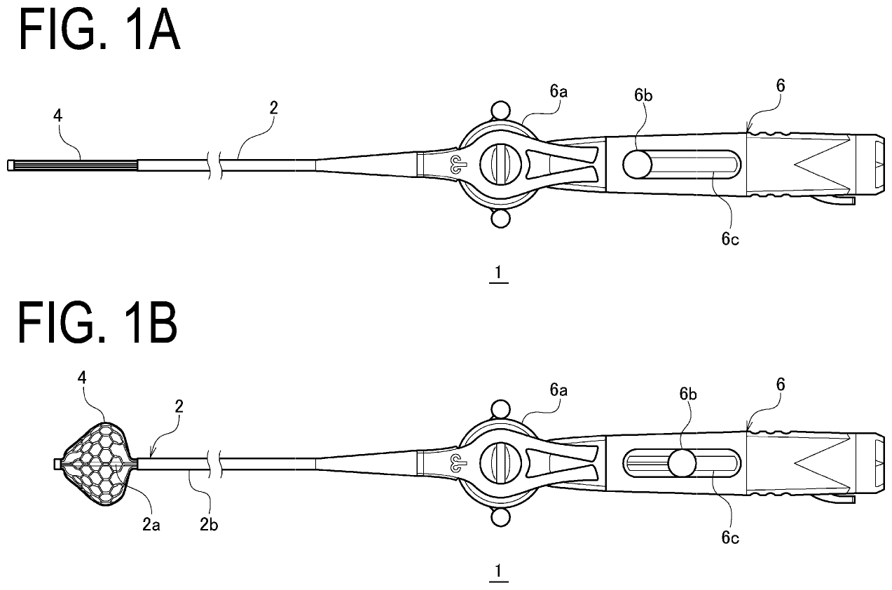

FIGS. 1A and 1B are side views of a catheter 1 according to an embodiment. FIG. 1A illustrates a state in which an electrode 4 is folded. FIG. 1B illustrates a state in which the electrode 4 is expanded. The catheter 1 includes a shaft 2 as an elongated body, the electrode 4, and a handle 6. “Elongated” in the present embodiment means that a ratio (first length/second length) of a first length in a longitudinal direction to a second length in a direction perpendicular to the longitudinal direction is 5 or more as an example. The present embodiment uses the shaft 2 as an example of the elongated body, but the elongated body may be an operating wire or the like.

The shaft 2 is made of a flexible tubular body, and at least the distal end side thereof is inserted into the body. The shaft 2 is made of known flexible materials including resin such as polyolefin, polytetrafluoroethylene, polyether block amide, or polyamide. The shaft 2 has a first length of, for example, from 600 mm to 1800 mm. The shaft 2 includes an inner shaft 2a having a tubular shape and an outer shaft 2b having a tubular shape. The inner shaft 2a is slidably inserted into the outer shaft 2b. A guide wire (not illustrated) or the like is inserted into the inner shaft 2a.

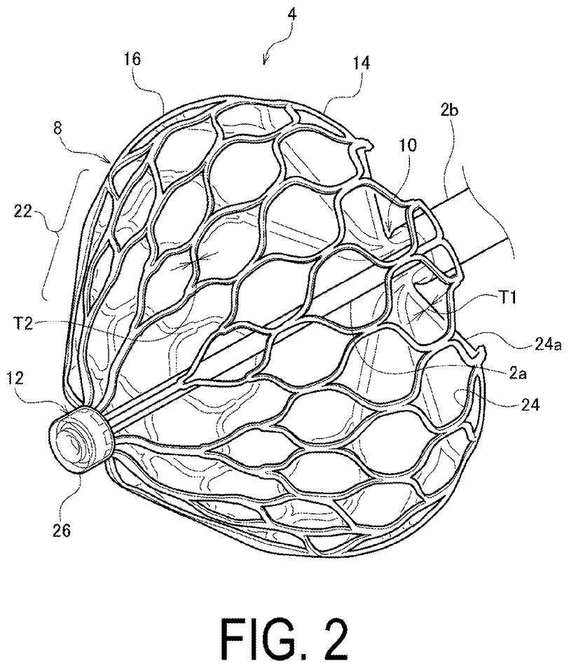

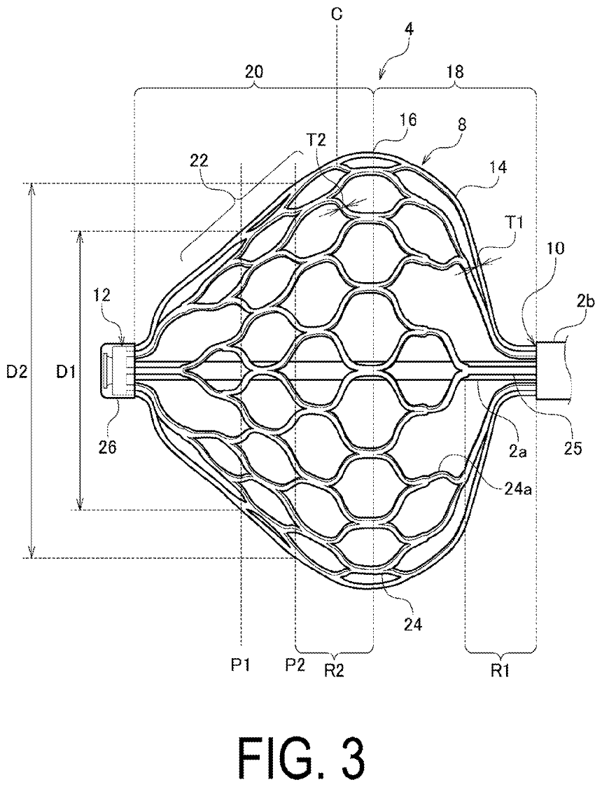

The electrode 4 is provided around an axis of the shaft 2 in a region on the distal end side (distal tip side) of the shaft 2, that is, a portion of the shaft 2 to be inserted into the body. The electrode 4 is substantially spherical. Hereinafter, the side of the catheter 1 or the shaft 2 on which the electrode 4 is provided is simply referred to as the “distal end side” as appropriate. The distal end of the inner shaft 2a protrudes from the distal end of the outer shaft 2b. The electrode 4 is disposed on a portion of the inner shaft 2a protruding from the distal end of the outer shaft 2b. More specifically, a distal end portion of the electrode 4 is fixed to a distal end of the inner shaft 2a, and the proximal end portion of the electrode 4 is fixed to the distal end of the outer shaft 2b (see also FIGS. 2 and 3). The shape, material, and the like of the electrode 4 will be described later in detail.

The handle 6 is provided on the proximal end side (proximal extremity side) of the shaft 2. Hereinafter, the side of the catheter 1 or the shaft 2 on which the handle 6 is provided is simply referred to as the “proximal end side” as appropriate. The handle 6 is disposed outside the body in using the catheter 1 and is gripped or operated by an operator. The handle 6 includes a deflection operating portion 6a and an expansion operating portion 6b. The operator can change the direction of the distal end side of the shaft 2 by operating the deflection operating portion 6a. Since the structure of the deflection operating portion 6a is known, detailed description thereof is omitted.

The operator can expand or deploy the electrode 4 in a folded state in a direction intersecting the axis of the shaft 2 by operating the expansion operating portion 6b. The electrode 4 in an expanded state can be folded by operating the expansion operating portion 6b. The expansion operating portion 6b is connected to the inner shaft 2a and is slidable along a guide rail 6c in an axial direction of the shaft 2 (a direction in which the axis of the shaft 2 extends or a lengthwise direction of the shaft 2, hereinafter simply referred to as “axial direction”). Sliding the expansion operating portion 6b located on the distal end side of the guide rail 6c as illustrated in FIG. 1A toward the proximal end side as illustrated in FIG. 1B displaces the inner shaft 2a toward the proximal end side with respect to the outer shaft 2b. This causes the distal end portion of the electrode 4 to approach the proximal end portion, causing the electrode 4 to swell in the direction intersecting the axis of the shaft 2. That is, the electrode 4 expands. A reverse operation, that is, sliding the expansion operating portion 6b toward the distal end side displaces the inner shaft 2a toward the distal end side with respect to the outer shaft 2b, causing the distal end portion of the electrode 4 to be separated from the proximal end portion and the electrode 4 to contract. That is, the electrode 4 is folded.

Subsequently, the electrode 4 will be described in detail. FIG. 2 is a perspective view of the electrode 4. FIG. 3 is a side view of the electrode 4. FIGS. 2 and 3 illustrate the expanded state of the electrode 4. The electrode 4 in the expanded state includes an intermediate portion 8, a proximal end portion 10 and a distal end portion 12. The intermediate portion 8 at least partially includes a portion in which element wires 14 spread like a mesh. The intermediate portion 8 of the present embodiment is entirely composed of a mesh of the element wires 14. The proximal end portion 10 is a portion which is located closer to the proximal end side of the shaft 2 than the intermediate portion 8 and in which the element wires 14 gather. The proximal end portion 10 is fixed to the outer shaft 2b. The distal end portion 12 is a portion which is located closer to the distal end side than the intermediate portion 8 and in which the element wires 14 gather. The distal end portion 12 is fixed to the inner shaft 2a.

The intermediate portion 8 includes a maximum portion 16 where the outer dimension of the electrode 4 is maximized. The outer dimension of the electrode 4 is the dimension of the electrode 4 in the direction perpendicular to the axis of the shaft 2. When the element wires 14 are located on a perfect circle in the cross-section of the electrode 4 perpendicular to the axis of the shaft 2, the diameter of this perfect circle corresponds to the outer dimension of the electrode 4. When the element wires 14 are not located on the perfect circle, the length of the longest straight line of the straight lines connecting any two element wires 14 corresponds to the outer dimension of the electrode 4.

The maximum portion 16 is located closer to the proximal end side than the center C of the electrode 4 in the axial direction. The electrode 4 is divided into a proximal end-side region 18 from the proximal end portion 10 to the maximum portion 16 and a distal end-side region 20 from the maximum portion 16 to the distal end portion 12. In the distal end-side region 20, the outer dimension D1 of the electrode 4 at a first position P1 in the axial direction is smaller than the outer dimension D2 of the electrode 4 at a second position P2 located closer to the proximal end side than the first position P1. The first position P1 and the second position P2 can be appropriately set by the designer.

The electrode 4 of the present embodiment includes, in the distal end-side region 20, a tapered portion 22 whose outer dimension gradually decreases toward the distal end portion. The tapered portion 22 is provided on the entire circumference around the axis of the shaft 2. Thus, the electrode 4 of the present embodiment has a substantially conical shape the bottom surface of which swells toward the proximal end side. That is, the diameter of the electrode 4 gradually decreases from the maximum portion 16 toward the distal end portion 12. As an example, the tapered portion 22 occupies two-thirds or more of the distal end-side region 20 in the axial direction. The electrode 4 may have a shape of a prolate spheroid or the like.

In the first region R1 continuing from the proximal end portion 10 in the axial direction within the proximal end-side region 18 and the second region R2 continuing from the maximum portion 16 in the axial direction within the distal end-side region 20, the number of the element wires 14 in the first region R1 is less than the number of the element wires 14 in the second region R2. That is, the number of the element wires 14 appearing on the cross-section perpendicular to the axis of the shaft 2 in the first region R1 is less than the number of the element wires 14 appearing on the cross-section perpendicular to the axis of the shaft 2 in the second region R2. The thickness T1 of the element wires 14 in the first region R1 is larger than the thickness T2 of the element wires 14 in the second region R2. The first region R1 illustrated in FIG. 3 is part of the proximal end-side region 18, and the second region R2 is part of the distal end-side region 20. However, without being limited to this configuration, the ranges of the first region R1 and the second region R2 can be appropriately set by the designer. Although the distal end of the second region R2 coincides with the second position P2 in FIG. 3, configurations are not particularly limited to this configuration.

The electrode 4 includes a plurality of opening portions 24 defined by the element wires 14. The plurality of opening portions 24 corresponds to meshes. At least some of the opening portions 24a located in the proximal end-side region 18 extend to the proximal end portion 10. These opening portions 24a each have an open end 25 at the proximal end portion 10. In other words, the entire circumferences of the opening portions 24a are not surrounded by the element wires 14, and the opening portions 24a are open at the proximal end side.

The distal end portion 12 is covered with an insulating cap 26. A partial region that continues from the proximal end portion 10 and a partial region that continues from the distal end portion 12 in the electrode 4 are covered with an insulating coating (not illustrated). A region including the maximum portion 16 and the tapered portion 22 in the intermediate portion 8 is exposed without being covered with the insulating coating.

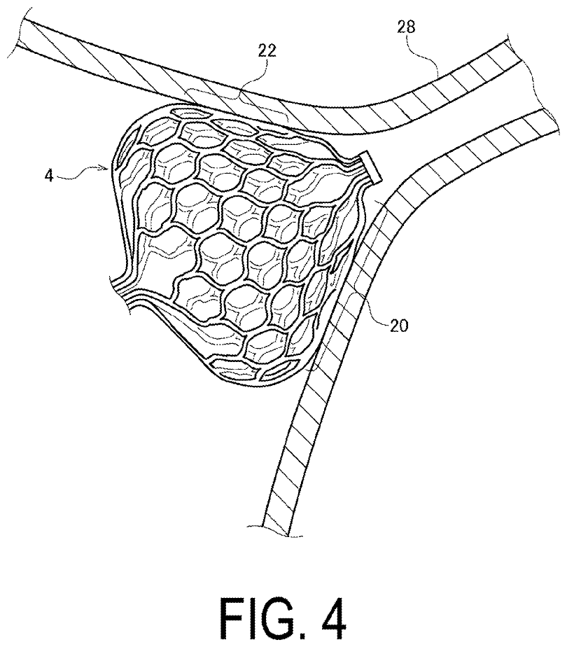

FIG. 4 is a view illustrating a usage state of the catheter 1. FIG. 4 only illustrates the electrode 4. The catheter 1 of the present embodiment is a catheter for pulsed electric field ablation (PFA). The catheter 1 is inserted into a body of a patient, for example, the inside of the heart, through the blood vessel, and is used for treatment of arrhythmia and the like. As an example, the electrode 4 is disposed at the entrance of a treatment site 28 that tapers in diameter toward the interior, and the distal end-side region 20 is mainly inserted into the treatment site 28. Examples of the treatment site 28 include a blood vessel such as a pulmonary vein and superior vena cava, and a pouched portion (recess) such as an auricle.

The electrode 4 is connected to an external power source (not illustrated) via a lead wire (not illustrated) that is inserted through the shaft 2 or laid on the outer surface of the shaft 2. When power is supplied to the electrode 4 from the external power source, the electrode 4 generates a pulsed electric field. This causes cells located near the electrode 4 to die, treating atrial fibrillation or the like. PFA has shorter time of output than conventional radio-frequency ablation (RFA), allowing transmission of excessive heat to other tissues through the treatment site 28 to be suppressed. Therefore, damage to tissues other than the treatment site 28 can be suppressed. As a result, complications such as phrenic nerve palsy and esophageal fistula can be suppressed.

In the electrode 4 of the present embodiment, the maximum portion 16 is shifted from the center C to the proximal end side. Thus, the distal end-side region 20 can be enlarged. This can increase, in inserting the electrode 4 into the tapered treatment site 28, the contact area between the electrode 4 and the inner wall of the treatment site 28. As a result, the area that can be treated with the catheter 1 can be increased. This can improve the performance of the catheter 1.

In the distal end-side region 20, the outer dimension D1 at the first position P1 is smaller than the outer dimension D2 at the second position P2. The tapered portion 22 is provided in the distal end-side region 20. This allows the electrode 4 to easily have surface contact with the inner wall of the treatment site 28. As a result, the usability of the catheter 1 can be further improved.

In the electrode 4 of the present embodiment, the number of the element wires 14 is smaller, and the thickness thereof is larger, in the first region R1 than in the second region R2. Thickening the element wire 14 in the first region R1 more than the element wires 14 in the second region R2 can increase the rigidity of the proximal end-side region 18. Reducing the number of the element wires 14 in the first region R1 less than the number of the element wires 14 in the second region R2 can thicken the element wires 14 while suppressing increase in the outer dimension of the proximal end-side region 18 in the folded state of the electrode 4. In adjusting the position of the electrode 4 by insertion and removal of the catheter 1 or in pressing the electrode 4 against the treatment site 28, a large load is applied to the proximal end-side region 18, and thus the proximal end-side region 18 is likely to be deformed. In response to this, increasing the rigidity of the proximal end-side region 18 to suppress deformation can facilitate position adjustment of the electrode 4 and pressing of the electrode 4 against the treatment site 28. This can improve the performance of the catheter 1.

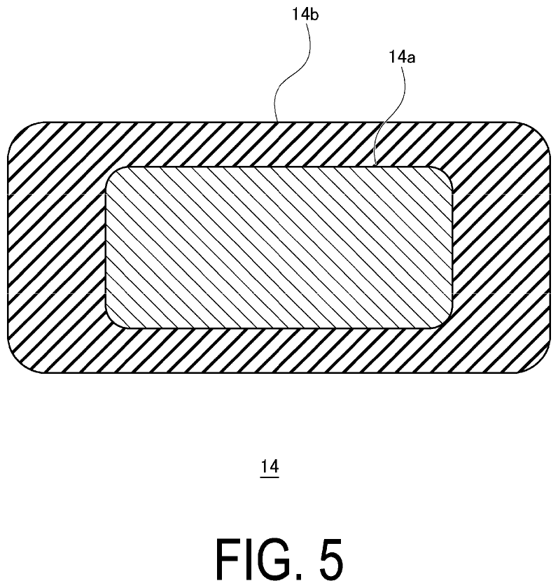

FIG. 5 is a cross-sectional view of the element wire 14 that constitutes the electrode 4. The cross-sectional shape of the element wire 14 is not limited to a rectangle. The element wire 14 that constitutes the electrode 4 includes a core wire 14a and a coating 14b covering the core wire 14a. The core wire 14a contains a first metal. The first metal has a standard electrode potential lower than a standard electrode potential of hydrogen. The coating 14b contains a second metal. The second metal has a standard electrode potential higher than the standard electrode potential of hydrogen and is less likely to occlude hydrogen than the first metal. Preferably, the core wire 14a contains the first metal as a main component, and the coating 14b contains the second metal as a main component. In the present embodiment, “contain as a main component” means that the first metal accounts for 50 atomic % or more and preferably 70 atomic % or more of all the components constituting the core wire 14a. Similarly, it means that the second metal accounts for 50 atomic % or more and preferably 70 atomic % or more of all the components constituting the coating 14b. The content of each component in each of the core wire 14a and the coating 14b is the average value of the content at any multiple measurement points.

In the present embodiment, “less likely to occlude hydrogen” means that the hydrogen occlusion amount (mass %) of the second metal is smaller than the hydrogen occlusion amount of the first metal.

In the present embodiment, the first metal contains Ni and/or Ti. The second metal contains Au and/or Pt. For example, the core wire 14a is made of Ni—Ti alloy, and the coating 14b is an Au-plated layer or a Pt-plated layer.

In PFA, a current having a biphasic waveform is applied to the electrode 4. Thus, the polarity of the electrode 4 is alternately switched in units of microseconds (for example, 1 μs). In PFA, a higher voltage (for example, 2000 V) is applied to the electrode 4 than that in RFA. As a result of intensive studies on the catheter 1 for PFA, the present inventors have found that the electrode can be corroded by a high voltage applied in PFA. That is, in the case of an electrode composed only of the core wire 14a containing the first metal whose standard electrode potential is lower than the standard electrode potential of hydrogen, when the electrode becomes an anode, the constituent metal of the electrode may be eluted by electrolysis (thinning). When the electrode becomes a cathode, hydrogen generated by electrolysis penetrates into the constituent metal of the electrode, and thus the metal may be embrittled (hydrogen embrittlement).

The thinning or the hydrogen embrittlement of the electrode decreases the strength of the electrode. In response to this, in the electrode 4 of the present embodiment, the core wire 14a is covered with the coating 14b containing the second metal whose standard electrode potential is higher than the standard electrode potential of hydrogen. This can suppress the thinning of the electrode 4. The second metal is less likely to occlude hydrogen than the first metal. This can suppress the hydrogen embrittlement of the electrode 4. Therefore, the durability of the electrode 4 and thus the catheter 1 can be improved, and the performance of the catheter 1 can be improved.

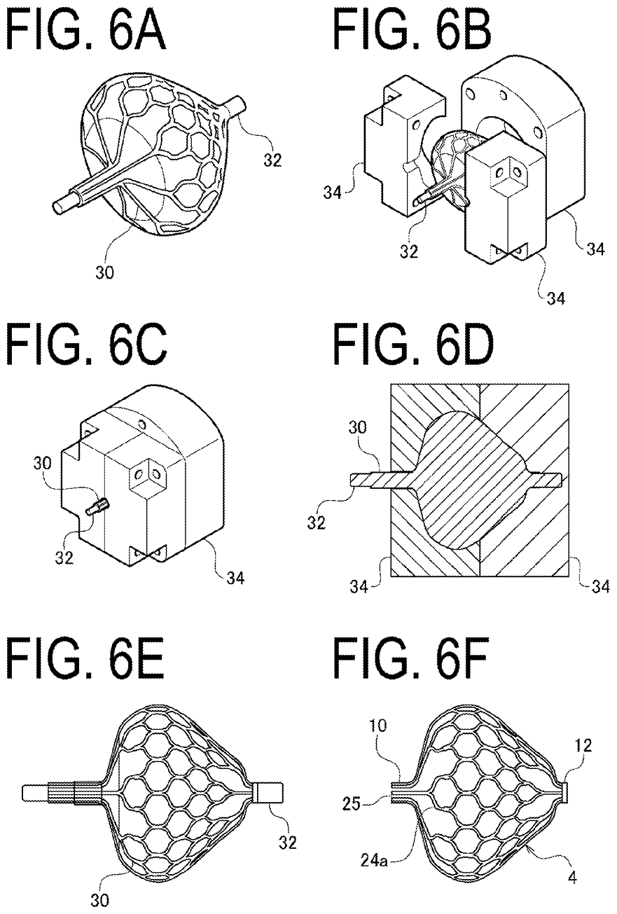

FIGS. 6A to 6F are diagrams each illustrating a manufacturing process of the electrode 4. First, grid-like cuts are made in a pipe 30 (for example, Ni—Ti pipe) containing the first metal by laser cutting or the like. As illustrated in FIG. 6A, the pipe 30 is then expanded and a first mold 32 having a substantially conical shape is inserted into the pipe 30. In this way, the pipe 30 is maintained in the expanded state.

Next, as illustrated in FIG. 6B, the pipe 30 and the first mold 32 are placed in a cavity of a second mold 34. As a result, as illustrated in FIGS. 6C and 6D, the expanded pipe 30 is sandwiched between the first mold 32 and the second mold 34. In this state, the pipe 30 is heated at a high temperature of, for example, about 500° C. for predetermined time. This applies shape memory processing to the pipe 30. As illustrated in FIG. 6E, the second mold 34 is then removed. Subsequently, the first mold 32 is removed from the pipe 30, and the pipe 30 is coated with the second metal. As a result, as illustrated in FIG. 6F, the electrode 4 whose expanded shape is memorized is obtained.

The opening portions 24a located in the proximal end-side region 18 each have an open end 25 at the proximal end portion 10. Thus, the proximal end portion 10 can be opened wider than the distal end portion 12. In this way, the first mold 32 can be easily removed from the electrode 4. Therefore, the electrode 4 can be manufactured more easily. In addition. providing the opening portions 24a having the open end 25 in the proximal end-side region 18 can increase the contact area between the treatment site 28 and the electrode 4 compared to providing the opening portion 24a in the distal end-side region 20.

The shape of the electrode 4 described above means the shape of the portion composed of the element wires 14, that is, the shape of the element wires 14 themselves. Thus, in the electrode 4 in which a member such as a contrast marker is joined to the element wires 14, the shape of the portion excluding the joined member corresponds to the shape of the electrode 4 in the present embodiment.

The embodiment of the present disclosure has been described in detail. The embodiment described above is merely a specific example for carrying out the present disclosure. The content of the embodiment is not intended to limit the technical scope of the present disclosure. Many design changes such as changes, additions, and deletions of components can be made in the scope that does not depart from the spirit of the present disclosure specified in the claims. A new embodiment with design changes has effects of combined embodiments and variations thereof. In the embodiment described above, the content in which such design changes can be made has been emphasized with expressions such as “of the present embodiment” or “in the present embodiment,” but design changes are also possible even in the content without such an expression. Any combination of components included in each embodiment is also effective as an aspect of the present disclosure. Hatching in sections of the drawings does not limit the material of a hatched object.

The embodiment may be identified by the items described below.

### [Item 1]

A catheter (1), including:
an elongated body (2) at least a distal end side of which is to be inserted into a body; and
an electrode (4) provided around an axis of the elongated body (2) and expandable in a direction intersecting the axis, wherein
the electrode (4) in the expanded state includes an intermediate portion (8) at least partially including a portion in which element wires (14) spread like a mesh, a proximal end portion (10) which is located closer to a proximal end side of the elongated body (2) than the intermediate portion (8) and in which the element wires (14) gather, and a distal end portion (12) which is located closer to the distal end side than the intermediate portion (8) and in which the element wires (14) gather,
the intermediate portion (8) includes a maximum portion (16) where an outer dimension of the electrode (4) is maximized, and
the maximum portion (16) is located closer to the proximal end side than a center (C) of the electrode (4) in an axial direction of the elongated body (2).

### [Item 2]

The catheter (1) according to item 1, wherein in a distal end-side region (20) from the maximum portion (16) to the distal end portion (12), an outer dimension (D1) of the electrode (4) at a first position (P1) in the axial direction of the elongated body (2) is smaller than an outer dimension (D2) of the electrode (4) at a second position (P2) closer to the proximal end side than the first position (P1).

### [Item 3]

The catheter (1) according to item 2, wherein the electrode (4) includes, in the distal end-side region (20), a tapered portion (22) whose outer dimension gradually decreases toward the distal end portion (12).

### [Item 4]

A catheter (1), including:
an elongated body (2) at least a distal end side of which is to be inserted into a body; and
an electrode (4) provided around an axis of the elongated body (2) and expandable in a direction intersecting the axis, wherein
the electrode (4) in the expanded state includes an intermediate portion (8) at least partially including a portion in which element wires (14) spread like a mesh, a proximal end portion (10) which is located closer to a proximal end side of the elongated body (2) than the intermediate portion (8) and in which the element wires (14) gather, and a distal end portion (12) which is located closer to the distal end side than the intermediate portion (8) and in which the element wires (14) gather,
the intermediate portion (8) includes a maximum portion (16) where an outer dimension of the electrode (4) is maximized,
the electrode (4) includes a proximal end-side region (18) from the proximal end portion (10) to the maximum portion (16) and a distal end-side region (20) from the maximum portion (16) to the distal end portion (12), and
in a first region (R1) continuing from the proximal end portion (10) in an axial direction of the elongated body (2) within the proximal end-side region (18) and a second region (R2) continuing from the maximum portion (16) in the axial direction within the distal end-side region (20), the number of the element wires (14) in the first region (R1) is less than the number of the element wires (14) in the second region (R2), and a thickness of the element wires (14) in the first region (R1) is larger than a thickness of the element wires (14) in the second region (R2).

### [Item 5]

The catheter (1) according to item 4, wherein
the electrode (4) includes a plurality of opening portions (24) defined by the element wires (14), and
at least some of the opening portions (24a) located in the proximal end-side region (18) extend to the proximal end portion (10) and each have an open end (25) at the proximal end portion (10).

### [Item 6]

A catheter (1) for pulsed electric field ablation, including:
an elongated body (2) at least a distal end side of which is to be inserted into a body; and
an electrode (4) that is provided on the elongated body (2) and generates a pulsed electric field, wherein
the electrode (4) includes a core wire (14a) and a coating (14b) covering the core wire (14a),
the core wire (14a) contains a first metal whose standard electrode potential is lower than a standard electrode potential of hydrogen, and
the coating (14b) contains a second metal whose standard electrode potential is higher than the standard electrode potential of hydrogen and which is less likely to occlude hydrogen than the first metal.

### [Item 7]

The catheter (1) according to item 6, wherein
the first metal contains Ni and/or Ti, and
the second metal contains Au and/or Pt.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter, comprising:
an elongated body at least a distal end side of which is to be inserted into a body; and
an electrode provided around an axis of the elongated body and expandable in a direction intersecting the axis, wherein
the electrode in an expanded state includes an intermediate portion at least partially including a portion in which element wires spread like a mesh, a proximal end portion which is located closer to a proximal end side of the elongated body than the intermediate portion and in which the element wires gather, and a distal end portion which is located closer to the distal end side than the intermediate portion and in which the element wires gather,
the intermediate portion includes a maximum portion where an outer dimension of the electrode is maximized, and
the maximum portion is located closer to the proximal end side than a center of the electrode in an axial direction of the elongated body.

2. The catheter according to claim 1, wherein in a distal end-side region from the maximum portion to the distal end portion, an outer dimension of the electrode at a first position in the axial direction of the elongated body is smaller than an outer dimension of the electrode at a second position closer to the proximal end side than the first position.

3. The catheter according to claim 2, wherein the electrode includes, in the distal end-side region, a tapered portion whose outer dimension gradually decreases toward the distal end portion.

4. A catheter, comprising:
an elongated body at least a distal end side of which is to be inserted into a body; and
an electrode provided around an axis of the elongated body and expandable in a direction intersecting the axis, wherein
the electrode in the expanded state includes an intermediate portion at least partially including a portion in which element wires spread like a mesh, a proximal end portion which is located closer to a proximal end side of the elongated body than the intermediate portion and in which the element wires gather, and a distal end portion which is located closer to the distal end side than the intermediate portion and in which the element wires gather,
the intermediate portion including a maximum portion where an outer dimension of the electrode is maximized,
the electrode includes a proximal end-side region from the proximal end portion to the maximum portion and a distal end-side region from the maximum portion to the distal end portion, and
in a first region continuing from the proximal end portion in the axial direction of the elongated body within the proximal end-side region and a second region continuing from the maximum portion in an axial direction within the distal end-side region, the number of the element wires in the first region is less than the number of the element wires in the second region, and a thickness of the element wires in the first region is larger than a thickness of the element wires in the second region.

5. The catheter according to claim 4, wherein
the electrode includes a plurality of opening portions defined by the element wires, and
at least some of the opening portions located in the proximal end-side region extend to the proximal end portion and each have an open end at the proximal end portion.

6. A catheter for pulsed electric field ablation, comprising:
an elongated body at least a distal end side of which is to be inserted into a body; and
an electrode that is provided on the elongated body and generates a pulsed electric field, wherein
the electrode includes a core wire and a coating covering the core wire,
the core wire contains a first metal whose standard electrode potential is lower than a standard electrode potential of hydrogen, and
the coating contains a second metal whose standard electrode potential is higher than the standard electrode potential of hydrogen and which is less likely to occlude hydrogen than the first metal.

7. The catheter according to claim 6, wherein
the first metal contains Ni and/or Ti, and
the second metal contains Au and/or Pt.
